(19) United States
(12) Patent Application Publication
(10) Pub. No.: US 2019/0274986 A1
(43) Pub. Date: Sep. 12, 2019

(54) STERILE INJECTABLE COMPOSITION COMPRISING MELPHALAN

(71) Applicant: CADILA HEALTHCARE LIMITED, Ahmedabad (IN)

(72) Inventors: Kannan Essakimuthu MUTHAIYYAN, Ahmedabad (IN); Debjani Manoj SINGH, Ahmedabad (IN); Nirav Ishwarlal KHATRI, Ahmedabad (IN); Omprakash Ganesh Ram SHARMA, Ahmedabad (IN)

(21) Appl. No.: 16/292,639

(22) Filed: Mar. 5, 2019

(30) Foreign Application Priority Data
Mar. 6, 2018 (IN) 201821008248

(51) Int. Cl. A61K 31/198; A61K 47/24; A61K 47/26; A61K 47/32; A61K 9/08; A61K 9/19; A61K 9/00

(52) U.S. Cl. CPC A61K 31/198; A61K 47/24; A61K 47/26; A61K 9/0019; A61K 9/08; A61K 9/19; A61K 47/32

(57) ABSTRACT

The present invention relates to sterile injectable compositions comprising melphalan and one or more phospholipids. The present invention also relates to processes for preparing the sterile injectable compositions.

### FIELD OF THE INVENTION

The field of the present invention relates to sterile injectable compositions comprising melphalan and one or more phospholipids. The present invention also relates to processes for preparing the sterile injectable compositions.

### BACKGROUND OF THE INVENTION

Cancer is a group of diseases involving abnormal cell growth with the potential to invade or spread to other parts of the body. Chemotherapy is a category of cancer treatment that uses at least one anti-cancer drug (chemotherapeutic agent) as part of a standardized chemotherapy regimen.

Melphalan is an alkylating drug, indicated for use as a high-dose conditioning treatment prior to hematopoietic progenitor (stem) cell transplantation in patients with multiple myeloma. Melphalan is also indicated for the palliative treatment of patients with multiple myeloma for whom oral therapy is not appropriate. The chemical name of melphalan hydrochloride is 4-[bis(2-chloroethyl)amino]-L-phenylalanine hydrochloride. Melphalan is practically insoluble in water, but freely soluble in 1 N HCl and methanol. The marketed product Evomela® (melphalan) for injection, of Spectrum Pharmaceuticals Inc., is supplied as a sterile white to off-white lyophilized powder in a single-dose vial for intravenous use. Each vial contains 50 mg melphalan free base equivalent to 56 mg melphalan hydrochloride and 2700 mg Betadex Sulfobutyl Ether Sodium, NF.

The effect of sulfobutylether beta-cyclodextrin on the kidney appears to be dose-dependent and temporally determined. In animal trials, single-dose studies up to 2000 mg/kg i.v. sulfobutylether beta-cyclodextrin showed renal tolerance. Evidence of renal cellular toxicity was observed in studies with repeated administration over 1-6 months at sulfobutylether beta-cyclodextrin doses of ≥160 mg/kg/day, although even at much higher doses renal function was not impaired (Luke et al., J Pharm Sci 2010; 99: 3291-3301).

Marc A von Mach et al. (BMC Clin Pharmacol. 2006; 6: 6) mentions that there are chances of accumulation of sulphobutylether beta-cyclodextrin sodium in patients. It further mentions that systematic clinical investigations are required to prevent potential harm to patients due to an accumulation of sulphobutylether beta-cyclodextrin sodium.

There exists a need of an alternative, improved and stable injectable composition which enables efficacious and safe parenteral delivery of melphalan.

### SUMMARY OF THE INVENTION

In one general aspect, the present invention provides a sterile injectable composition comprising melphalan and one or more pharmaceutically acceptable excipients.

In another general aspect, the sterile injectable composition comprises melphalan, one or more phospholipids and one or more stabilizers.

In another general aspect, the sterile injectable composition may be in the form of a clear solution comprising melphalan-phospholipid micelles. The micelles may be melphalan-DMPG micelles.

In another general aspect, the sterile injectable composition may be in the form of a stable lyophilized powder comprising melphalan and one or more phospholipids.

In another general aspect, the present invention provides a process for preparing the sterile injectable composition.

In another general aspect, the invention provides a method of treating cancer by administering the sterile injectable composition of the present invention to the individual in need thereof.

The details of one or more embodiments of the invention are set forth in the description below. Other features, objects and advantages of the invention will be apparent from the description.

### DETAILED DESCRIPTION OF THE INVENTION

The present invention provides a sterile injectable composition comprising melphalan and one or more pharmaceutically acceptable excipients.

The sterile injectable composition of the present invention may be in the form of a stable lyophilized powder or a stable, clear, aqueous solution. The sterile injectable composition of the invention is not a powder for suspension, liquid suspension or nanoparticulate dispersion.

The sterile injectable composition of the present invention may be administered via parenteral route, for example, intravenous (I.V.), subcutaneous (S.C.) or intramuscular (I.M.).

The sterile injectable composition of the present invention may provide one or more advantages like, providing a safe composition by solubilizing the drug without usage of ingredients such as sulfobutylether beta-cyclodextrin, polysorbate 80 or Cremophor®, providing a stable, clear, aqueous solution suitable for I.V. administration compatible with routinely used i.v. sets and having improved stability, efficacy, and safety profile. Polysorbate 80 and cremophor are reported as the cause of hypersensitivity reactions while beta-cyclodextrin derivatives have been known to cause certain unwanted effects.

The sterile injectable composition of the present invention does not comprise cholesterol or cholesterol derivatives (for example, sodium cholesteryl sulphate etc.), lecithin or its derivative, phosphatidylcholines and/or polysorbate 80.

The sterile injectable composition may comprise melphalan and one or more phospholipids. The composition may comprise melphalan, 1,2-dimyristoyl-sn-glycero-3-phosphorylglycerol (DMPG), N-(carbonyl-methoxypolyethyleneglycol 2000)-1,2-distearoyl-sn-glycero-3-phosphoethanolamine (mPEG₂₀₀₀-DSPE), or any combination thereof. The composition may further comprise one or more stabilizers.

In one embodiment, the sterile injectable composition is a stable, clear, aqueous solution comprising one or more drugs and one or more pharmaceutically acceptable excipients. The stable, clear, aqueous solution may comprise one or more drugs, one or more phospholipids and one or more stabilizers. The stable, clear, aqueous solution may comprise drug micelles and is lyophilizable. The stable, clear, aqueous solution of the present invention does not comprise bilayered micelles (bicelles).

In one embodiment, the sterile injectable composition is a stable lyophilized powder comprising melphalan and one or more pharmaceutically acceptable excipients. The stable lyophilized powder may comprise melphalan, one or more phospholipids and one or more stabilizers.

In another embodiment, the invention also provides a sterile injectable composition comprising melphalan which is therapeutically equivalent to the commercially available melphalan formulation marketed under the trade name Evomela®.

The sterile injectable composition comprising melphalan of the invention may provide value of mean Cₘₐₓ (maximum concentration of drug in plasma, achieved after administration) between 3000 ng/mL and 9000 ng/mL, for example, between 3500 ng/mL and 8500 ng/mL, between 4000 ng/mL and 8000 ng/mL, between 4500 ng/mL and 7500 ng/mL, between 5000 ng/mL and 7000 ng/mL, between 5500 ng/mL and 6500 ng/mL or about 6000 ng/mL, when the sterile injectable composition comprising melphalan is administered as intravenous infusion to a human at a dose of 100 mg melphalan/m² for 30 minutes.

The sterile injectable composition comprising melphalan of the invention may provide value of mean AUC_{0-inf} (area under the curve for a plot of concentration of drug in plasma vs. time infinite) between 3,00,000 min-ng/mL and 6,00,000 min-ng/mL, for example, between 3,25,000 min-ng/mL and 5,75,000 min-ng/mL, between 3,50,000 min-ng/mL and 5,50,000 min-ng/mL, between 3,75,000 min-ng/mL and 5,25,000 min-ng/mL, between 4,00,000 min-ng/mL and 5,00,000 min-ng/mL or about 4,50,000 min-ng/mL, when the sterile injectable composition comprising melphalan is administered as intravenous infusion to a human at a dose of 100 mg melphalan/m² for 30 minutes.

In one embodiment, the stable lyophilized powder for injection comprises melphalan and one or more phospholipids.

In another embodiment, the stable lyophilized powder for injection comprises melphalan and one or more micelle forming phospholipids, wherein the phospholipid is having fatty acid carbon chain length of 16 carbons or less, for example, 16 carbons, 14 carbons or 12 carbons.

In another embodiment, the stable lyophilized powder comprises melphalan and one or more phospholipid. The stable lyophilized powder may further comprise one or more stabilizers.

In another embodiment, the stable lyophilized powder comprises melphalan, 1,2-dimyristoyl-sn-glycero-3-phosphorylglycerol (DMPG) and N-(Carbonyl-methoxypolyethylene glycol 2000)-1,2-distearoyl-sn-glycero-3-phosphoethanolamine (mPEG₂₀₀₀-DSPE). The stable lyophilized powder may further comprise polyvinylpyrrolidone.

In another embodiment, the stable lyophilized powder comprises melphalan, 1,2-dimyristoyl-sn-glycero-3-phosphorylglycerol (DMPG), N-(Carbonyl-methoxypolyethylene glycol 2000)-1,2-distearoyl-sn-glycero-3-phosphoethanolamine (mPEG₂₀₀₀-DSPE) and polyvinylpyrrolidone. The weight ratio of DMPG to mPEG₂₀₀₀-DSPE may be between about 10:1 and 1:10, for example, between 5:1 and 1:8, between 2:1 and about 1:5, about 3:1, about 1:1, about 1:2, about 1:3 or about 1:4.

In another embodiment, there is provided a vial comprising stable lyophilized powder comprising about 50 mg melphalan (equivalent to 56 mg melphalan HCl), about 70 mg DMPG Na, about 200 mg N-(Carbonyl-methoxypolyethyleneglycol 2000)-1,2-distearoyl-sn-glycero-3-phosphoethanolamine sodium (mPEG₂₀₀₀-DSPE Na) and about 500 mg polyvinylpyrrolidone K12 (PVP K12).

In another embodiment, there is provided a vial comprising stable lyophilized powder comprising about 50 mg melphalan (equivalent to 56 mg melphalan HCl), about 80 mg DMPG Na, about 20 mg N-(Carbonyl-methoxypolyethyleneglycol 2000)-1,2-distearoyl-sn-glycero-3-phosphoethanolamine sodium (mPEG₂₀₀₀-DSPE Na) and about 80 mg sucrose.

The amount of DMPG present in a vial comprising stable lyophilized powder comprising melphalan may be between about 10 mg to about 200 mg, for example, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg or about 190 mg. The weight ratio of melphalan to DMPG may be between 2:1 and 1:20, for example, between 1:1 and 1:10, between 1:1 and 1:5, about 1:1.4, about 1:2 or about 1:3.

The amount of mPEG₂₀₀₀-DSPE present in a vial comprising stable lyophilized powder comprising melphalan may be between about 25 mg and about 500 mg, for example, about 50 mg, about 75 mg, about 100 mg, about 110 mg, about 120 mg, about 130 mg, about 140 mg, about 175 mg, about 200 mg, about 250 mg, about 300 mg, about 350 mg, about 400 mg, about 450 mg or about 475 mg. The weight ratio of melphalan to mPEG₂₀₀₀-DSPE may be between 2:1 and 1:10, for example, between 1:1 and 1:8, about 1:2, about 1:4 or about 1:6.

The amount of PVP present in a vial comprising stable lyophilized powder comprising melphalan may be between about 25 mg and about 1000 mg, for example, about 50 mg, about 100 mg, about 150 mg, about 200 mg, about 250 mg, about 300 mg, about 400 mg, about 450 mg, about 500 mg, about 550 mg, about 600 mg, about 650 mg, about 700 mg, about 800 mg, about 900 mg or about 1000 mg. The weight ratio of melphalan to PVP may be between 1:1 and 1:50, for example, between 1:1 and 1:30, about 1:5, about 1:10 or about 1:15.

The amount of sucrose present in a vial comprising stable lyophilized powder comprising melphalan may be between about 40 mg and about 2000 mg, for example, about 50 mg, about 100 mg, about 150 mg, about 200 mg, about 250 mg, about 300 mg, about 400 mg, about 450 mg, about 500 mg, about 550 mg, about 600 mg, about 650 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg or about 1900 mg. The weight ratio of melphalan to sucrose may be between 1:10 and 1:50, for example, between 1:15 and 1:35, about 1:16, about 1:20, about 1:30, about 1:35, about 1:40 or about 1:45. The weight ratio of melphalan to stabilizer(s) may be between 1:1 and 1:60, for example, between 1:5 and 1:40, about 1:15, about 1:25, about 1:30, about 1:35, about 1:40, about 1:45, about 1:50 or about 1:55.

In one embodiment, the stable lyophilized powder is powder for solution. The stable lyophilized powder may be reconstituted using an appropriate quantity of aqueous vehicle (e.g. water for injection, 5% dextrose or 0.9% NaCl) to provide a stable, clear, aqueous reconstituted solution. The reconstituted solution may be diluted using an appropriate quantity of diluent (e.g. water for injection, 5% dextrose or 0.9% NaCl) to provide a stable, clear, aqueous diluted solution.

In another embodiment, the sterile injectable composition is a stable, clear, aqueous solution comprising one or more drugs and one or more phospholipids. The solution of the invention does not form any precipitate after storage for relevant time period, for example, for 6 hours, for 8 hours, for 10 hours, for 12 hours, for 18 hours, for 24 hours or for 48 hours, at temperature 2° C.-8° C. or at 25° C.±2° C. temperature. The stable, clear, aqueous solution of the invention does not comprise liposome or nanoparticle or any other particulate drug delivery system.

In another embodiment, the sterile injectable composition is a stable, clear, aqueous solution comprising one or more drugs, one or more phospholipids and one or more stabilizers.

In another embodiment, the stable, clear, aqueous solution comprises melphalan and one or more phospholipids. The stable, clear, aqueous solution may comprises melphalan, 1,2-dimyristoyl-sn-glycero-3-phosphorylglycerol (DMPG) and N-(Carbonyl-methoxypolyethylene glycol 2000)-1,2-distearoyl-sn-glycero-3-phosphoethanolamine (mPEG₂₀₀₀-DSPE). The stable, clear, aqueous solution may further comprise polyvinylpyrrolidone. The solution may be lyophilizable. The solution may be used for parenteral administration. The solution for parenteral administration may be in the form of 100% water based solution and does not comprise any other solvent or liquid vehicle.

In another embodiment, the present invention provides a stable, clear, aqueous solution comprising melphalan, (mPEG₂₀₀₀-DSPE), and polyvinylpyrrolidone, wherein the solution may have pH between about 2 and about 7, for example, about 3, about 4, about 5 or about 6.

In another embodiment, the present invention provides a stable, clear, aqueous solution comprising melphalan, DMPG, mPEG₂₀₀₀-DSPE and one or more stabilizers, wherein the concentration of melphalan may be between about 0.01 mg/mL and about 100 mg/mL, for example, between about 0.05 mg/mL and about 10 mg/mL, about 0.1 mg/mL, about 0.45 mg/mL or about 5 mg/mL. The amount of DMPG present in the diluted solution comprising melphalan may be between about 0.1 mg/mL and about 20 mg/mL, for example, about 0.2 mg/mL, about 0.4 mg/mL, about 0.63 mg/mL, about 1 mg/mL, about 2 mg/mL, about 3 mg/mL, about 4 mg/mL, about 5 mg/mL, about 6 mg/mL, about 7 mg/mL, about 8 mg/mL, about 10 mg/mL, about 12 mg/mL or about 15 mg/mL. The amount of mPEG₂₀₀₀-DSPE present in the diluted solution comprising melphalan may be between about 0.1 mg/mL and about 50 mg/mL, for example, about 0.5 mg/mL, about 1 mg/mL, about 1.5 mg/mL, about 1.8 mg/mL, about 2 mg/mL, about 3 mg/mL, about 5 mg/mL, about 10 mg/mL, about 15 mg/mL, about 20 mg/mL, about 25 mg/mL, about 30 mg/mL, about 40 mg/mL or about 50 mg/mL. The amount of PVP present in the diluted solution comprising melphalan may be between about 1 mg/mL and about 100 mg/mL, for example, about 2 mg/mL, about 3 mg/mL, about 4 mg/mL, about 4.5 mg/mL, about 5 mg/mL, about 6 mg/mL, about 7 mg/mL, about 8 mg/mL, about 9 mg/mL, about 10 mg/mL, about 20 mg/mL, about 30 mg/mL, about 40 mg/mL, about 45 mg/mL, about 50 mg/mL, about 60 mg/mL, about 70 mg/mL, about 80 mg/mL, about 90 mg/mL or about 95 mg/mL.

The stable, clear, aqueous solution may be in the form of micellar solution comprising melphalan, one or more phospholipids and one or more stabilizers.

### Definitions

As used herein, and unless otherwise specified, the term “stable” refers to the stability of the product or the composition comprising one or more drugs having sufficient physical and chemical stability for a relevant period of time under the specified storage conditions.

The term “physical stability” with respect to clear solution refers to maintenance of clear state without any drug precipitation, maintenance of color or colorless state and/or maintenance of dissolved oxygen level. The term “physical stability” with respect to lyophilized powder refers to maintenance of aesthetic appearance, absence of agglomerates, and maintenance of flowability, dispersibility and water content.

The term “chemical stability” relates to maintenance of drug-related impurities in terms of total impurities, known impurities, single maximum known impurity and single maximum unknown impurity, within the allowed limits by the regulatory agency.

Unless and otherwise mentioned, the term “melphalan” includes, but not limited to, melphalan base, melphalan hydrochloride and polymorphs thereof.

Unless and otherwise mentioned, the term “1,2-dimyristoyl-sn-glycero-3-phosphorylglycerol/DMPG” includes, but not limited to, 1,2-dimyristoyl-sn-glycero-3-phosphorylglycerol/DMPG base, 1,2-dimyristoyl-sn-glycero-3-phosphorylglycerol/DMPG sodium, or any other salts thereof.

Unless and otherwise mentioned, the term “N-(carbonyl-methoxypolyethyleneglycol 2000)-1,2-distearoyl-sn-glycero-3-phosphoethanolamine/mPEG₂₀₀₀-DSPE” includes, but not limited to, N-(carbonyl-methoxypolyethyleneglycol 2000)-1,2-distearoyl-sn-glycero-3-phosphoethanolamine/mPEG₂₀₀₀-DSPE base, N-(carbonyl-methoxypoly ethylene glycol 2000)-1,2-distearoyl-sn-glycero-3-phosphoethanolamine/mPEG₂₀₀₀-DSPE sodium, or any other salts thereof.

The stable lyophilized powder of the invention remains stable for commercially relevant time period after manufacturing, such as for about 1, 3, 6, 12, 18, 24 or 36 months, when it is kept in its original packaging under the specified storage conditions.

The reconstituted solution or diluted solution of the present invention which is prepared by mixing lyophilized powder and suitable liquid vehicle remains stable for relevant hold time period, such as for about 1, 3, 6, 8, 12, 18, 24 or 36 hours when it is kept under the specified storage conditions.

The diluted solution of the invention which is prepared by mixing the reconstituted solution and suitable liquid diluent and remains stable for relevant hold time period, such as for about 1, 3, 6, 8, 12, 18, 24 or 36 hours when it is kept under the specified storage conditions.

The term “lyophilizable” means a solution which is able to go under lyophilization process using conventional methods and provides lyophilized powder or cake upon lyophilization.

As used herein, the term “micelle” means an aggregate of surfactant and/or phospholipid molecules, normally spherical in shape, wherein the aggregate have hydrophobic center and hydrophilic periphery.

As used herein, the term “drug-phospholipid micelle” means a micelle formed by drug and phospholipid molecules, wherein drug molecules are present in solubilized form in the hydrophobic core of the micelle.

As used herein, the term “melphalan-DMPG micelle” means a micelle, comprising melphalan and DMPG molecules, wherein melphalan molecules are present in solubilized form in the hydrophobic core of the micelle.

As used herein, the term “melphalan-mPEG₂₀₀₀-DSPE micelle” means a micelle, comprising melphalan and mPEG₂₀₀₀-DSPE molecules, wherein melphalan molecules are present in solubilized form in the hydrophobic core of the micelle.

As used herein, the term “clear solution” means a solution which does not comprise any visible particulate matter, liposome or nanoparticles. The clear solution provides absorbance, when measured at 420 nm, not more than 0.1 AU (absorbance unit), for example, not more than 0.05 AU, not more than 0.04 AU or not more than 0.03 AU. The clear solution provides % transmittance, when measured at 650 nm, not less than 97%, for example, not less than 98%, not less than 99%, not less than 99.5%, not less than 99.6%, not less than 99.7% or not less than 99.8%.

As used herein, the term “powder for injection” means powder to prepare liquid composition suitable for parenteral administration. Such liquid composition may be prepared by mixing the powder with one or more of suitable liquid diluents such as water, dextrose, saline and the like.

As used herein, the term “buffer” means a chemical agent able to absorb a certain quantity of acid or base without undergoing a strong variation in the pH.

As used herein, the term “about” refers to encompass +/−20%, 15%, 10%, 5%, 2%, 1%, 0.5%, or 0.25% of the numerical value of the number with which it is being used.

### Abbreviations

RT=Room temperature
RH=Relative humidity
1M, 2M and 3M=1 month, 2 months and 3 months, respectively.

### ICH=International Conference on Harmonisation

ND=Not detected
NP=Not performed
NA=Not analyzed
CRT=Controlled room temperature
BQL=Below quantification level/limit
BDL=Below detection level/limit
HPLC=High performance liquid chromatography
KF titration=Karl Fischer titration
WFI=Water for injection
° C.=Degree Celsius (unit of temperature)
μ=Micrometer (unit of length)

| Impurity | IUPAC Name | |
|---|---|---|
| Type | of Impurity | Structure of Impurity |
| Monochloro Melphalan | 2-Amino-3-{4- [(2-chloroethyl)- 2-hydroxy-ethyl)- amino]-phenyl}- propionic acid | 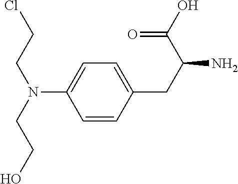 [SMILES: N[C@@H](Cc1ccc(N(CCO)CCCl)cc1)C(=O)O] |
| Melphalan dimer | 2-Amino-3-{4- [[2-(2-{4-[bis- (2-chloro-ethyl)- amino]-phenyl}- 1-carboxy-ehtil- amino)-ethyl]- (2-chloro-ethyl)- amino]-phenyl}- propionic acid | 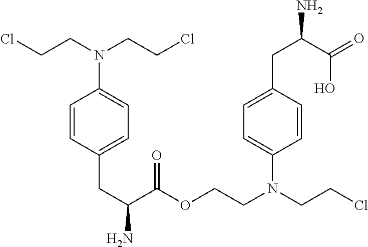 [SMILES: N[C@H](Cc1ccc(N(CCCl)CCOC(=O)[C@@H](N)Cc2ccc(N(CCCl)CCCl)cc2)cc1)C(=O)O] |
| Methoxy Melphalan | 2-Amino-3-(4- ((2-chloroethyl) (2-methoxyethyl)- amino)phenyl)- propionic acid | 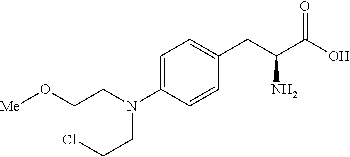 [SMILES: COCCN(CCCl)c1ccc(C[C@H](N)C(=O)O)cc1] |

The suitable pharmaceutically acceptable excipients for the composition of the present invention may include one or more of the pharmaceutically acceptable solvents, phospholipids, pH adjusting agents, stabilizers, buffers, cryoprotectants, preservatives, isotonicity adjusting agents, surfactants, and anti-oxidants.

Examples of solvents may include, but not limited to, water for injection, organic solvents or mixture of water for injection with one or more organic solvents. The examples of organic solvents are tert-butyl alcohol, dehydrated alcohol, acetonitrile, dimethyl sulfoxide, dimethyl acetamide, dimethylformamide, N-methyl-2-pyrrolidone (NMP), acetone, methanol, benzonitrile, isopropyl alcohol, propylene glycol, polyethylene glycol, glycerine and the like.

Examples of phospholipids may include, but not limited to, phosphatidylcholines such as 1,2-dimyristoyl-sn-glycero-3-phosphocholine (DMPC), 1,2-dioleoyl-sn-glycero-3-phosphocholine (DOPC) and the like; phosphatidylglycerols such as 1,2-dimyristoyl-sn-glycero-3-phosphorylglycerol sodium (DMPG Na), 1,2-dipalmitoyl-sn-glycero-3-phosphorylglycerol sodium (DPPG Na), 1,2-distearoyl-sn-glycero-3-phosphorylglycerol sodium (DSPG Na), 1,2-dioleoyl-sn-glycero-3-phosphorylglycerol sodium (DOPG Na) and the like; phosphatidylserines such as 1,2-dimyristoyl-sn-glycero-3-phosphoserine sodium (DMPS Na), 1,2-dipalmitoyl-sn-glycero-3-phosphoserine sodium (DPPS Na), 1,2-distearoyl-sn-glycero-3-phosphoserine sodium (DSPS Na), 1,2-dioleoyl-sn-glycero-3-phosphoserine sodium (DOPS Na) and the like; phosphatidic acids such as 1,2-dimyristoyl-sn-glycero-3-phosphatidic acid sodium (DMPA Na), 1,2-dipalmitoyl-sn-glycero-3-phosphatidic acid sodium (DPPA Na), 1,2-distearoyl-sn-glycero-3-phosphatidic acid, sodium (DSPA Na), 1,2-dioleoyl-sn-glycero-3-phosphatidic acid sodium (DOPA Na) and the like; phosphatidylethanolamines such as 1,2-dimaristoyl-sn-glycero-3-phosphoethanolamine (DMPE), 1,2-dioleoyl-sn-glycero-3-phosphoethanolamine (DOPE) and the like; methoxy conjugated phospholipids such as N-(Carbonyl-methoxypolyethylene glycol 2000)-1,2-distearoyl-sn-glycero-3-phosphoethanolamine sodium (mPEG₂₀₀₀-DSPE Na), N-(Carbonyl-methoxypolyethylene glycol 5000)-1,2-distearoyl-sn-glycero-3-phosphoethanolamine sodium (mPEG₅₀₀₀-DSPE Na), N-(Carbonyl-methoxypolyethylene glycol 2000)-1,2-dipalmitoyl-sn-glycero-3-phosphoethanolamine sodium (mPEG₂₀₀₀-DPPE Na), N-(Carbonyl-methoxypolyethyleneglycol 5000)-1,2-dipalmitoyl-sn-glycero-3-phosphoethanolamine sodium (mPEG₅₀₀₀-DPPE Na) and the like, or any combination thereof. The amount of the phospholipid present in the composition may vary and depends on the nature and type of the drug as well as amount of drug to be administered, nature and type of the phospholipid, type of interaction between drug and phospholipid molecules in the aqueous phase, etc. For example, amount of dimyristoyl phosphatidylglycerol (DMPG) may be such that the solution contains concentration of DMPG between 1 mg/mL and 50 mg/mL. For example, amount of N-(Carbonyl-methoxypolyethylene glycol 2000)-1,2-distearoyl-sn-glycero-3-phosphoethanolamine (mPEG₂₀₀₀-DSPE) may be such that the solution contains concentration of mPEG₂₀₀₀-DSPE between 1 mg/mL and 50 mg/mL.

Examples of pH adjusting agents may include, but not limited to, sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, sodium carbonate, sodium bicarbonate, triethanolamine, or any combination thereof.

Examples of stabilizers may include, but not limited to, polyvinylpyrrolidone, polyvinyl alcohol, polyethylene glycol, diethanolamine, ferric chloride, inositol, sodium gluconate, sucrose, mannitol, creatinine, glycerine, niacinamide, sodium saccharin, sodium caprylate, arginine, methionine, cysteine and the like, or any combination thereof.

Examples of buffers may include, but not limited to, acetate (e.g. sodium acetate etc.), citrate (e.g. citric acid/sodium citrate etc.), phosphate (e.g. monobasic sodium phosphate, dibasic sodium phosphate etc.), carbonate, or any combination thereof.

Examples of cryoprotectants may include, but not limited to, sucrose, lactose, mannitol, polyethylene glycol, polyvinylpyrrolidone, or any combination thereof.

Examples of preservatives may include, but not limited to, chlorobutanol, benzalkonium chloride, methyl paraben, propyl paraben, benzoic acid, sodium benzoate, sorbic acid, benzethonium chloride, cetyl pyridinium chloride, benzyl bromide, benzyl alcohol, phenylmercury nitrate, phenylmercury acetate, thiomersal, merthiolate, chlorhexidine, phenylethyl alcohol, quaternary ammonium chloride, sodium benzoate, sodium propionate, or any combination thereof.

Examples of isotonicity adjusting agents may include, but not limited to, sodium chloride, potassium chloride, calcium chloride, magnesium chloride, glucose, sucrose, dextrose, mannitol, glycerol, or any combination thereof.

Examples of suitable pharmaceutically acceptable surfactants may include, but not limited to, amphoteric, non-ionic, cationic or anionic molecules. Suitable surfactants may include, but not limited to, polysorbates (e.g. tween 80 etc.), poloxamer (poloxamer 188), sodium lauryl sulfate, lauryl dimethyl amine oxide, docusate sodium, cetyl trimethyl ammonium bromide (CTAB), polyethoxylated alcohols, polyoxyethylene sorbitan, octoxynol, polyoxyl lauryl ether, polyoxyethylene vegetable-based fatty ethers derived from lauryl, cetyl, stearyl and oleyl alcohols, bile salts (such as sodium deoxycholate and sodium cholate), polyoxyl castor oil, nonylphenol ethoxylate, lecithin, polyoxyethylene surfactants, polyethylene glycol esters, glycol esters of fatty acids, monoalkanolamine condensates, polyoxyethylene fatty acid amides, quaternary ammonium salts, polyoxyethylene alkyl and alicyclic amines, polyoxyethylene, sorbitan monolaurate and stearate, Cremophor® (polyethoxylated castor oil), Solutol® (ethylene oxide/12-hydroxy stearic acid), tyloxapol, or any combination thereof.

Examples of suitable pharmaceutically acceptable anti-oxidants may include, but not limited to, butylated hydroxytoluene (BHT), butylated hydroxyanisole (BHA), and propyl gallate (PG), monothioglycerol, ascorbic acid, citric acid, tartaric acid, sodium ascorbate, erythorbic acid, potassium metabisulfite, sodium metabisulfite, propionic acid, sodium formaldehyde sulphoxylate, reduced glutathione, thiourea, cysteine, n-acetylcysteine, methionine, sodium sulfite, alkyl gallate, including propyl gallate, vitamin E, or other tocopherol analogs, including tocopherol acetate or TPGS, or any combination thereof.

The sterile injectable compositions of the present invention may have improved physical and/or chemical stability.

The stable lyophilized powder composition of the present invention may retain at least 90% potency (assay) of the drug after storage for more than 6 months, for example, for 12 months, 18 months, 24 months or 36 months, at 2° C.-8° C.

The stable lyophilized powder composition of the present invention may retain at least 90% potency (assay) of the drug after storage for more than 6 months, for example, for 12 months, 18 months, 24 months or 36 months, at 25° C.±2° C. temperature and 60% RH (relative humidity).

The stable lyophilized powder composition of the present invention may retain at least 90% potency (assay) of the drug after storage for more than 6 months, for example, for 12 months, 18 months, 24 months or 36 months, at 40° C.±2° C. and 75% RH.

The stable, clear, aqueous solution of the present invention may retain at least 90% potency (assay) of the drug after storage for 1 hour or more, for example, storage for 2 hours, 4 hours, 6 hours, 8 hours, 12 hours, 18 hours, 24 hours, 36 hours or 48 hours, at 2° C.-8° C.

The stable, clear, aqueous solution of the present invention may retain at least 90% potency (assay) of the drug after storage for 1 hour or more, for example, storage for 2 hours, 4 hours, 6 hours, 8 hours, 12 hours, 18 hours, 24 hours, 36 hours or 48 hours, at 25° C.±2° C. temperature.

In one embodiment, there is provided a stable lyophilized powder comprising melphalan, which remains stable and compliant as per regulatory requirements for impurities for respective product, for more than 2 months, for example, for 3 months, for 6 months, for 12 months, for 24 months or for 36 months, when stored at 25° C.±2° C. temperature and 60% RH or at 2° C.-8° C. temperature.

In one embodiment, there is provided a stable, clear, aqueous solution comprising melphalan, which remains stable and compliant as per regulatory requirements for impurities for respective product, for more than 4 hours, for example, for 8 hours, for 12 hours, for 18 hours, 24 hours or for 36 hours, when stored at 2° C.-8° C. temperature or at 25° C.±2° C. temperature.

The stable, clear, aqueous solution comprising melphalan does not form precipitate and remains physically stable up to 6 hours, 8 hours, 10 hours, 12 hours, 18 hours, 24 hours, 36 hours or 48 hours when stored at 2° C.-8° C. temperature or at 25° C.±2° C. temperature.

In another embodiment of the invention, there is provided a stable lyophilized powder comprising melphalan which does not contain more than 1% of monochloro melphalan impurity, does not contain more than 1% of methoxy melphalan impurity, does not contain more than 1% of melphalan dimer impurity, does not contain more than 1% of the single maximum unknown impurity and/or does not contain more than 10% (for example, not more than 8%, 6%, 4%, or 2%) of the total impurities when stored at 2° C.-8° C. temperature, for more than 1 months, for example, for 2 months, for 3 months, for 6 months, for 12 months, for 18 months, for 24 months or for 36 months.

In another embodiment of the invention, there is provided a stable lyophilized powder comprising melphalan which does not contain more than 1% of monochloro melphalan impurity, does not contain more than 1% of methoxy melphalan impurity, does not contain more than 1% of melphalan dimer impurity, does not contain more than 1% of the single maximum unknown impurity and/or does not contain more than 10% (for example, not more than 8%, 6%, 4%, or 2%) of the total impurities when stored at 25° C.±2° C. temperature and 60% RH, for more than 1 months, for example, for 2 months, for 3 months, for 6 months, for 12 months, for 18 months, for 24 months or for 36 months.

In another embodiment of the invention, there is provided a stable, clear, aqueous solution comprising melphalan which does not contain more than 1% of monochloro melphalan impurity, does not contain more than 1% of methoxy melphalan impurity, does not contain more than 1% of melphalan dimer impurity, does not contain more than 1% of the single maximum unknown impurity and/or does not contain more than 10% (for example, not more than 8%, 6%, 4%, or 2%) of the total impurities when stored at 2° C.-8° C. temperature, for more than 0.5 hour, for example, for 1 hour, for 2 hours, for 3 hours, for 4 hours, for 5 hours, for 6 hours, for 7 hours, for 8 hours, for 10 hours, for 12 hours, for 18 hours, for 24 hours or for 48 hours.

In another embodiment of the invention, there is provided a stable, clear, aqueous solution comprising melphalan which does not contain more than 1% of monochloro melphalan impurity, does not contain more than 1% of methoxy melphalan impurity, does not contain more than 1% of melphalan dimer impurity, does not contain more than 1% of the single maximum unknown impurity and/or does not contain more than 10% (for example, not more than 8%, 6%, 4%, or 2%) of the total impurities when stored at 25° C.±2° C. temperature, for more than 0.5 hour, for example, for 1 hour, for 2 hours, for 3 hours, for 4 hours, for 5 hours, for 6 hours, for 7 hours, for 8 hours, for 10 hours, for 12 hours, for 18 hours, for 24 hours or for 48 hours.

The stable, clear, aqueous solution comprising melphalan does not form precipitate and remains physically stable for up to 0.5 hour, 1 hour, 2 hours, 3 hours, 4 hours, 5 hours, 6 hours, 7 hours, 8 hours, 10 hours, 12 hours, 18 hours, 24 hours, 36 hours or 48 hours when stored at 2° C.-8° C. temperature or at 25° C.±2° C. temperature.

In one embodiment, the present invention provides a process for preparing the lyophilized powder comprising one or more drugs and one or more phospholipids. The process includes the steps of (a) preparing solution comprising one or more solvents and one or more phospholipids; (b) adding drug to the solution obtained at step (a); and (c) lyophilizing so obtained solution of step (b) to obtain lyophilized powder. So obtained lyophilized powder may be reconstituted using an appropriate quantity of one or more of water for injection, 5% dextrose or normal saline solution (0.9% NaCl), to provide a stable, clear, aqueous reconstituted solution, for parenteral administration. The reconstituted solution may be further diluted using an appropriate quantity of one or more of water for injection, 5% dextrose or normal saline solution (0.9% NaCl), to provide a stable, clear, aqueous diluted solution, for parenteral administration.

In another embodiment, the present invention provides a process for preparing the lyophilized powder comprising melphalan and DMPG. The process includes the steps of (a) preparing solution comprising DMPG Na, water for injection and one or more organic solvents; (b) adding melphalan into the solution of step (a); and (c) lyophilizing so obtained solution mixture of step (b) to obtain lyophilized powder. Additionally, PVP and/or sodium chloride may be added at step (a), (b) or (c). Additionally, mPEG₂₀₀₀-DSPE may be added at step (a). So obtained lyophilized powder may be reconstituted using an appropriate quantity of one or more of water for injection, 5% dextrose or normal saline solution (0.9% NaCl), to provide a stable, clear, aqueous reconstituted solution, for parenteral administration. The reconstituted solution may be further diluted using an appropriate quantity of one or more of water for injection, 5% dextrose or normal saline solution (0.9% NaCl), to provide a stable, clear, aqueous diluted solution, for parenteral administration.

The following examples are for the purpose of illustration of the invention only and are not intended in any way to limit the scope of the present invention.

### Example 1

TABLE 1

| Ingredients | Quantity/mL | |
|---|---|---|
| Melphalan HCl | 5.6 | mg |
| 1,2-dimyristoyl-sn-glycero-3-phosphorylglycerol | 6.5 | mg |
| sodium (DMPG Na) | | |
| N-(Carbonyl-methoxypolyethyleneglycol 2000)- | 25 | mg |
| 1,2-distearoyl-sn-glycero- | | |
| 3-phosphoethanolamine sodium | | |
| (mPEG₂₀₀₀-DSPE Na) | | |
| Polyvinylpyrrolidone K12 (PVP K12) | 25 | mg |
| Sodium Chloride | 4 | mg |
| Sodium hydroxide | q.s. to pH 4.0 | |
| Hydrochloric acid | q.s. to pH 4.0 | |
| Tert-Butyl alcohol | 0.7 | mL |
| Dehydrated Alcohol | 0.05 | mL |

| | |
|---|---|
| Water for Injection (WFI) | q.s. 1 mL |

### Process

### Manufacturing Steps:

### (a) Ready for Lyophilization Solution

A solvent mixture composed of appropriate quantities of tert-butyl alcohol, water for injection and dehydrated alcohol was prepared in their volume ratio of 70:25:05.

To the obtained solvent mixture, accurately weighed DMPG Na and mPEG₂₀₀₀-DSPE Na were added at room temperature under stirring and continued to stir till it became clear solution. Further to the obtained solution, accurately weighed polyvinylpyrrolidone and sodium chloride were added under stirring and continued to stir till it became clear solution.

Then after, pH was adjusted using appropriate quantities of hydrochloric acid and sodium hydroxide to have pH about 6.

The volume of so obtained aqueous solution was made up with water for injection and filtered through 0.22μ filter. (Batch size: 200 mL)

### (b) Lyophilization

Thus obtained ready for lyophilization solution at step (a) was filled into 50 mL glass vials, in the required quantity so that each vial contains 50 mg of melphalan. So obtained glass vials were lyophilized to obtain lyophilized powder.

### (c) Reconstitution

### Reconstituted Solution

Thus obtained lyophilized powder in the above step (b) was reconstituted using water for injection to provide reconstituted solution having 2.5 mg/mL melphalan.

### (d) Dilution

### Diluted Solution

Thus obtained reconstituted solution in above step (c) was further diluted using an appropriate quantity of normal saline solution (0.9% NaCl), to provide diluted solution having 0.45 mg/mL melphalan

The reconstituted solution obtained in above mentioned step (c) and diluted solution obtained in above mentioned step (d) were tested for their physical stability and results are reported in below Table 2 and Table 3.

TABLE 2
Physical stability of reconstituted solution,
obtained at step (c) of Example 1

| Reconstitution | | |
|---|---|---|
| vehicle | Concentration | CRT |
| Water for | 2.5 mg/mL | Clear solution |
| injection | melphalan | at 24 hour |

TABLE 3
Physical stability of diluted solution,
obtained at step (d) of Example 1

| Dilution | | |
|---|---|---|
| vehicle | Concentration | CRT |
| Normal saline | 0.45 mg/mL | Clear solution |
| solution (0.9% NaCl) | melphalan | at 24 hour |

### Example 2

TABLE 4

| Ingredients | Quantity/mL | |
|---|---|---|
| Melphalan HCl | 5.6 | mg |
| 1,2-dimyristoyl-sn-glycero-3-phosphorylglycerol | 7 | mg |
| sodium (DMPG Na) | | |
| N-(Carbonyl-methoxypolyethyleneglycol 2000)- | 20 | mg |
| 1,2-distearoyl-sn- | | |
| glycero-3-phosphoethanolamine sodium | | |
| (mPEG₂₀₀₀-DSPE Na) | | |
| Polyvinylpyrrolidone K12 (PVP K12) | 50 | mg |
| Sodium Chloride | 6 | mg |
| Sodium hydroxide | q.s. to pH 4.0 | |
| Hydrochloric acid | q.s. to pH 4.0 | |
| Tert-Butyl alcohol | 0.7 | mL |
| Dehydrated Alcohol | 0.05 | mL |

| | |
|---|---|
| Water for Injection (WFI) | q.s. 1 mL |

### Process

Formula, batch size and process for the example 2 is similar to the example 1, except the amount of ingredients were used as per the Table 4.

The reconstituted solution obtained in the step (c) and diluted solution obtained in the step (d) of the example 2, were tested for their physical and chemical stability and results are reported in below Table 5 through Table 8.

TABLE 5
Physical stability of reconstituted solution,
obtained at step (c) of Example 2

| Reconstitution vehicle | Concentration | CRT |
|---|---|---|
| Water for | 5 mg/mL | Clear to slight bluish |
| injection | melphalan | solution at 5 hours |

TABLE 6
Physical stability of diluted solution
obtained at step (d) of Example 2

| Dilution vehicle | Concentration | CRT |
|---|---|---|
| Normal saline | 0.45 mg/mL | Clear solution |
| solution (0.9% NaCl) | melphalan | at 5 hours |

TABLE 7
Chemical stability of reconstituted solution,
obtained at step (c) of Example 2

| Concentration | | CRT | CRT |
|---|---|---|---|
| 5 mg/mL melphalan | Initial | 1 hour | 2 hours |
| Assay of melphalan | 91.1 | 89.2 | 88.5 |

TABLE 8
Chemical stability of diluted solution,
obtained at step (d) of Example 2

| Concentration | | CRT | CRT | CRT |
|---|---|---|---|---|
| 0.45 mg/mL melphalan | Initial | 1 hour | 2 hours | 4 hours |
| Assay of melphalan | 100 | 94.35 | 92.27 | 92.39 |

### Example 3

TABLE 9

| Ingredients | Quantity/mL | |
|---|---|---|
| Melphalan hydrochloride (56 mg) equivalent to | 2.80 | mg |
| anhydrous melphalan (50 mg) | | |
| 1.,2-dimyristoyl-sn-glycero-3-phosphorylglycerol | 3.25 | mg |
| sodium (DMPG Na) | | |
| N-(Carbonyl-methoxypolyethyleneglycol 2000)- | 12.50 | mg |
| 1,2-distearoyl-sn- | | |
| glycero-3-phosphoethanolamine sodium | | |
| (mPEG₂₀₀₀-DSPE Na) | | |
| Polyvinylpyrrolidone K12 (PVP K12) | 12.50 | mg |
| Sodium chloride | 2.00 | mg |
| Sodium hydroxide | q.s. to pH 4.00 | |
| Hydrochloric acid | q.s. to pH 4.00 | |
| Tert-butyl alcohol* | 0.70 | mL |
| Ethanol* | 0.05 | mL |

| | |
|---|---|
| Water for injection* | q.s. 1.00 mL |

*These vehicles will evaporate during freeze drying and will not be the part of final formulation.

Packaging details: Glass vial: 50 mL clear colorless type I glass vial. Rubber stopper: 20 mm bromobutyl grey slotted rubber stopper. Flip off aluminum Seal: 20 mm flip off seal.

### Process

### Manufacturing Steps:

### (a) Ready for Lyophilization Solution

A solvent mixture composed of appropriate quantities of tert-butyl alcohol:WFI:dehydrated alcohol was prepared in their volume ratio of 70:25:05.

To the obtained solvent mixture, accurately weighed DMPG Na and mPEG₂₀₀₀-DSPE Na were added at room temperature under stirring and continued to stir till it became clear solution. Further to the obtained solution, accurately weighed polyvinylpyrrolidone and sodium chloride were added under stirring and continued to stir till it became clear solution. Further to the obtained solution, accurately weighed drug was added under stirring and continued to stir till it became clear solution.

Then after, pH was adjusted using appropriate quantities of hydrochloric acid and sodium hydroxide to have pH about 4.

The volume of so obtained aqueous solution was made up with water for injection and filtered through 0.22μ filter. (Batch size: 1450 mL)

### (b) Lyophilization

Thus obtained ready for lyophilization solution at step (a) was filled into 50 mL glass vials in the required quantity so that each vial contains 50 mg of melphalan. So obtained glass vials were lyophilized to obtain lyophilized cake.

The lyophilized powder composition obtained in the step (b) of example 3 was tested for its initial stability as well as for stability after storage for 1 month, 2 months, 3 months, 6 months, and 9 months at 2° C.-8° C. temperature, and results are reported in below Table 10.

TABLE 10

| | | Storage Condition | | | | | |
|---|---|---|---|---|---|---|---|
| | | 2° C.-8° C. | | | | | |
| Tests | | Initial | 1 M | 2 M | 3 M | 6 M | 9 M |
| Description | | A white | A white | A white | A white | A white | A white |
| | | lyophilized | lyophilized | lyophilized | lyophilized | lyophilized | lyophilized |
| | | cake | cake | cake | cake | cake | cake |
| Assay of Melphalan by | | 98.500 | 100.600 | 103.300 | 101.400 | 101.500 | 102.400 |
| HPLC | | | | | | | |
| (Unit: % of label amount) | | | | | | | |
| Related | Monochloro | 0.040 | 0.050 | 0.040 | 0.050 | 0.030 | 0.040 |
| Substances | Melphalan | | | | | | |
| by HPLC | Methoxy | ND | ND | ND | 0.020 | 0.040 | 0.010 |
| (Unit: %) | Melphalan | | | | | | |
| | Melphalan | 0.040 | 0.050 | 0.090 | 0.060 | 0.080 | 0.200 |
| | Dimer | | | | | | |
| | Single | 0.080 | 0.130 | 0.130 | 0.110 | 0.100 | 0.100 |
| | maximum | | | | | | |
| | unknown | | | | | | |
| | impurity | | | | | | |
| | Total | 0.190 | 0.250 | 0.320 | 0.310 | 0.270 | 0.400 |
| | Impurities | | | | | | |

| | | | | | | |
|---|---|---|---|---|---|---|
| pH | 4.450 | 4.510 | 4.780 | 4.700 | 4.720 | 4.810 |
| Reconstitution Time | 4.000 | 3.200 | 3.500 | 3.200 | 3.000 | 4.200 |
| (Unit: minutes) | | | | | | |
| Water Content | 0.410 | NP | NP | 0.200 | 0.220 | NP |
| determination by KF | | | | | | |
| titration | | | | | | |
| (Unit: % w/w of the | | | | | | |
| lyophilized powder | | | | | | |
| composition) | | | | | | |

The lyophilized powder composition obtained in the step (b) of example 3 was tested for its initial stability as well as for stability after storage for 1 month, 2 months, 3 months, and 6 months at 25° C.±2° C. temperature and 60% RH, and results are reported in below Table 11.

TABLE 11

| | | Storage Condition | | | | |
|---|---|---|---|---|---|---|
| | | 25° C. ± 2° C. temperature and 60% RH | | | | |
| Tests | | Initial | 1 M | 2 M | 3 M | 6 M |
| Description | | A white | A white | A white | A white | A white |
| | | lyophilized | lyophilized | lyophilized | lyophilized | lyophilized |
| | | cake | cake | cake | cake | cake |
| Assay of Melphalan by | | 98.500 | 102.100 | 98.500 | 100.700 | 100.600 |
| HPLC | | | | | | |
| (Unit: % of label amount) | | | | | | |
| Related | Monochloro | 0.040 | 0.050 | 0.040 | 0.060 | 0.030 |
| Substances | Melphalan | | | | | |
| by HPLC | Methoxy | ND | ND | ND | 0.020 | 0.010 |
| (Unit: %) | Melphalan | | | | | |
| | Melphalan | 0.040 | 0.150 | 0.290 | 0.320 | 0.540 |
| | Dimer | | | | | |
| | Single | 0.080 | 0.180 | 0.140 | 0.110 | 0.090 |
| | maximum | | | | | |
| | unknown | | | | | |
| | impurity | | | | | |
| | Total | 0.190 | 0.380 | 0.500 | 0.560 | 0.750 |
| | Impurities | | | | | |

| | | | | | |
|---|---|---|---|---|---|
| pH | 4.450 | 4.580 | 4.720 | 4.710 | 4.730 |
| Reconstitution Time | 4.000 | 3.600 | 3.800 | 3.500 | 3.500 |
| (Unit: minutes) | | | | | |
| Water Content | 0.410 | NP | NP | 0.300 | 0.430 |
| determination by KF | | | | | |
| titration | | | | | |
| (Unit: % w/w of the | | | | | |
| lyophilized powder | | | | | |
| composition) | | | | | |

The lyophilized powder composition obtained in the step (b) of example 3 was stored at 2° C.-8° C. temperature for 1 month, 2 months, 3 months, 6 months and 9 months. The initial as well as said storage samples of lyophilized powder composition obtained in the step (b) of example 3 were reconstituted using water for injection to provide 2.5 mg/mL melphalan reconstituted solution. Thus obtained reconstituted solution was tested for its physical stability and results are reported in Table 12.

TABLE 12

| | Storage Condition | | | | | |
|---|---|---|---|---|---|---|
| | 2° C.-8° C. | | | | | |
| Tests | Initial | 1 M | 2 M | 3 M | 6 M | 9 M |
| Absorbance | 0.038 | 0.026 | 0.042 | 0.029 | 0.042 | 0.039 |
| (Unit: AU) | | | | | | |
| % | 97.400 | 99.200 | 98.300 | 97.900 | 97.600 | 97.200 |
| Transmittance | | | | | | |
| (Unit: %) | | | | | | |

The lyophilized powder composition obtained in the step (b) of example 3 was stored at 25° C.±2° C. temperature and 60% RH±5% RH for 1 month, 2 months, 3 months and 6 months. The initial as well as said storage samples of lyophilized powder composition obtained in the step (b) of example 3 were reconstituted using water for injection to provide 2.5 mg/mL melphalan reconstituted solution. Thus obtained reconstituted solution was tested for its physical stability and results are reported in Table 13.

TABLE 13

| Storage Condition | 25° C. ± 2° C. temperature and 60% RH | | | | |
|---|---|---|---|---|---|
| Tests | Initial | 1 M | 2 M | 3 M | 6 M |
| Absorbance | 0.038 | 0.033 | 0.039 | 0.040 | 0.070 |
| (Unit: AU) | | | | | |
| % Transmittance | 97.400 | 98.800 | 98.700 | 97.800 | 97.700 |
| (Unit: %) | | | | | |

### Example 4

TABLE 14

| Ingredients | Quantity/mL | |
|---|---|---|
| Melphalan hydrochloride (56 mg) equivalent to | 2.80 | mg |
| anhydrous melphalan (50 mg) | | |
| 1,2-dimyristoyl-sn-glycero-3-phosphorylglycerol | 3.00 | mg |
| sodium (DMPG Na) | | |
| N-(Carbonyl-methoxypolyethyleneglycol 2000)- | 7.00 | mg |
| 1,2-distearoyl-sn- | | |
| glycero-3-phosphoethanolamine sodium | | |
| (mPEG₂₀₀₀-DSPE Na) | | |
| Polyvinylpyrrolidone K12 (PVP K12) | 7.50 | mg |
| Sucrose | 75.00 | mg |
| Sodium hydroxide | q.s. to pH 4.00 | |
| Hydrochloric acid | q.s. to pH 4.00 | |
| Tert-butyl alcohol* | 0.70 | mL |

| | |
|---|---|
| Water for injection* | q.s. 1.00 mL |

*These vehicles will evaporate during freeze drying and will not be the part of final formulation.

Packaging details: Glass vial: 50 mL clear colorless type I glass vial. Rubber stopper: 20 mm bromobutyl grey slotted rubber stopper. Flip off aluminum Seal: 20 mm flip off seal.

### Process

### Manufacturing Steps:

### (a) Ready for Lyophilization Solution

A solvent mixture composed of appropriate quantities of tert-butyl alcohol:WFI was prepared in their volume ratio of 70:30.

To the obtained solvent mixture, accurately weighed DMPG Na and mPEG₂₀₀₀-DSPE Na were added at room temperature under stirring and continued to stir till it became clear solution. Further to the obtained solution, accurately weighed polyvinylpyrrolidone and sucrose were added under stirring and continued to stir till it became clear solution.

Further to the obtained solution, accurately weighed drug was added under stirring and continued to stir till it became clear solution.

Then after, pH was adjusted using appropriate quantities of hydrochloric acid and sodium hydroxide to have pH about 4.

The volume of so obtained aqueous solution was made up with water for injection and filtered through 0.22μ filter. (Batch size: 3000 mL)

### (b) Lyophilization

Thus obtained ready for lyophilization solution at step (a) was filled into 50 mL glass vials in the required quantity so that each vial contains 50 mg of melphalan. So obtained glass vials were lyophilized to obtain lyophilized cake.

### (c) Reconstitution

### Reconstituted Solution

Thus obtained lyophilized powder in above step (b) was reconstituted using appropriate quantities of 0.9% sodium chloride solution (saline) to provide reconstituted solution having 2.5 mg/mL melphalan.

The lyophilized powder composition obtained in the step (b) of example 4 was tested for its initial stability as well as for stability after storage for 1 month, 2 months, and 3 months, at 2° C.-8° C. as well as at 25° C.±2° C. temperature and 60% RH, and results are reported in below Table 15.

TABLE 15

| | | Storage Condition | | | | | | |
|---|---|---|---|---|---|---|---|---|
| | | | 2° C.-8° C. | | | 25° C. ± 2° C. temperature and 60% RH | | |
| Tests | | Initial | 1 M | 2 M | 3 M | 1 M | 2 M | 3 M |
| Description | | A white | A white | A white | A white | A white | A white | A white |
| | | lyophilized | lyophilized | lyophilized | lyophilized | lyophilized | lyophilized | lyophilized |
| | | cake | cake | cake | cake | cake | cake | cake |
| Assay of Melphalan by | | 99.000 | 98.900 | 98.900 | 99.200 | 95.700 | 95.000 | 95.000 |
| HPLC | | | | | | | | |
| (Unit: % of label amount) | | | | | | | | |
| Related | Monochloro | 0.260 | 0.240 | 0.240 | 0.260 | 0.2400 | 0.26 | 0.280 |
| Substances | Melphalan | | | | | | | |
| by HPLC | Melphalan | 0.200 | 0.260 | 0.300 | 0.350 | 1.200 | 2.200 | 3.000 |
| (Unit: %) | Dimer | | | | | | | |
| | Single | 0.050 | 0.040 | 0.040 | 0.070 | 0.050 | 0.090 | 0.100 |
| | maximum | | | | | | | |
| | unknown | | | | | | | |
| | impurity | | | | | | | |
| | Total | 0.630 | 0.650 | 0.760 | 0.870 | 1.700 | 2.900 | 3.800 |
| | Impurities | | | | | | | |

| | | | | | | | |
|---|---|---|---|---|---|---|---|
| pH | 4.370 | 4.390 | 4.020 | NP | 4.380 | 4.080 | NP |
| Reconstitution Time | 3.8 | 3.58 | 3.5 | NP | 3.5 | 3 | NP |
| (Unit: minutes) | | | | | | | |
| Water Content | 0.240 | 0.310 | NP | NP | 0.350 | NP | NP |
| determination by KF | | | | | | | |
| titration | | | | | | | |
| (Unit: % w/w of the | | | | | | | |
| lyophilized powder | | | | | | | |
| composition) | | | | | | | |

The reconstituted solution obtained at step (c) of example 4 was tested for its stability and results are reported in below Table 16.

TABLE 16
Stability of reconstituted solution (melphalan 2.5 mg/mL),
obtained at step (c) of Example 4

| Storage Condition | | 25° C. ± 2° C. temperature | | | |
|---|---|---|---|---|---|
| Tests | | Initial | 1 hour | 2 hours | 3 hours |
| Assay of Melphalan by HPLC | | 98.88 | 96.50 | 94.12 | 91.55 |
| (Unit: % of label amount) | | | | | |
| Related | Monochloro Melphalan | 0.52 | 2.00 | 3.40 | 4.90 |
| Substances | Melphalan Dimer | 0.35 | 1.20 | 2.00 | 2.70 |
| by HPLC | Single maximum | 0.04 | 0.04 | 0.08 | 0.18 |
| (Unit: %) | unknown impurity | | | | |
| | Total Impurities | 1.00 | 3.40 | 5.70 | 8.00 |

The lyophilized powder composition obtained in the step (b) of example 4 was stored at 2° C.-8° C. as well as at 25° C.±2° C. temperature and 60% RH±5% RH for 1 month, 2 months and 3 months. The initial as well as said storage samples of lyophilized powder composition obtained in the step (b) of example 4 were reconstituted using water for injection to provide 2.5 mg/mL melphalan reconstituted solution. Thus obtained reconstituted solution was tested for its physical stability and results are reported in Table 17.

TABLE 17

| | Storage Condition | | | | | | |
|---|---|---|---|---|---|---|---|
| | | | | | 25° C. ± 2° C. | | |
| | | | | | temperature | | |
| | | 2° C.-8° C. | | | and 60% RH | | |
| | | 1 | 2 | 3 | 1 | 2 | 3 |
| Tests | Initial | M | M | M | M | M | M |
| Absorbance | 0.033 | 0.033 | 0.036 | NP | 0.082 | 0.068 | NP |
| (Unit: AU) | | | | | | | |
| % Trans- | 98.570 | 98.760 | 98.050 | NP | 98.830 | 99.670 | NP |
| mittance | | | | | | | |
| (Unit: %) | | | | | | | |

### Example 5

TABLE 18

| Ingredients | Quantity/mL | |
|---|---|---|
| Melphalan hydrochloride (56 mg) equivalent to | 2.80 | mg |
| anhydrous melphalan (50 mg) | | |
| 1,2-dimyristoyl-sn-glycero-3-phosphorylglycerol | 4.00 | mg |
| sodium (DMPG Na) | | |
| N-(Carbonyl-methoxypolyethyleneglycol 2000)- | 10.00 | mg |
| 1,2-distearoyl-sn-glycero-3- | | |
| phosphoethanolamine | | |
| sodium (mPEG₂₀₀₀-DSPE Na) | | |
| Sucrose | 40.00 | mg |
| Sodium hydroxide | q.s. to pH 4.00 | |
| Hydrochloric acid | q.s. to pH 4.00 | |
| Tert-butyl alcohol* | 0.70 | mL |

| | |
|---|---|
| Water for injection* | q.s. 1.00 mL |

*These vehicles will evaporate during freeze drying and will not be the part of final formulation.

Packaging details: Glass vial: 50 mL clear colorless type I glass vial. Rubber stopper: 20 mm bromobutyl grey slotted rubber stopper. Flip off aluminum Seal: 20 mm flip off seal.

### Process

### Manufacturing Steps:

### (a) Ready for Lyophilization Solution

A solvent mixture composed of appropriate quantities of tert-butyl alcohol:WFI was prepared in their volume ratio of 70:30.

To the obtained solvent mixture, accurately weighed DMPG Na and mPEG₂₀₀₀-DSPE Na were added at room temperature under stirring and continued to stir till it became clear solution. Further to the obtained solution, accurately weighed sucrose was added under stirring and continued to stir till it became clear solution. Further to the obtained solution, accurately weighed drug was added under stirring and continued to stir till it became clear solution.

Then after, pH was adjusted using appropriate quantities of hydrochloric acid and sodium hydroxide to have pH about 4.

The volume of so obtained aqueous solution was made up with water for injection and filtered through 0.22μ filter. (Batch size: 100 mL)

### (b) Lyophilization

Thus obtained ready for lyophilization solution at step (a) was filled into 50 mL glass vials in the required quantity so that each vial contains 50 mg of melphalan. So obtained glass vials were lyophilized to obtain lyophilized cake.

### (c) Reconstitution

### Reconstituted Solution

Thus obtained lyophilized powder in above step (b) was reconstituted using appropriate quantities of 0.9% sodium chloride solution (saline) to provide reconstituted solution having 2.5 mg/mL melphalan.

The lyophilized powder composition obtained in the step (b) of example 5 was tested for its initial stability and results are reported in below Table 19.

TABLE 19

| Tests | | Initial |
|---|---|---|
| Assay of Melphalan by HPLC | | 94.1 |
| (Unit: % of label amount) | | |
| Related Substances | Monochloro Melphalan | 0.64 |
| by HPLC | Melphalan Dimer | 0.40 |
| (Unit: %) | Single maximum unknown impurity | 0.15 |
| | Total Impurities | 1.85 |

| | |
|---|---|
| pH | 4.56 |

The ready for lyophilization solution obtained in the step (a) of example 5 was tested for its stability and results are reported in the Table 20.

TABLE 20

| Storage Condition | | 2° C.-8° C. | |
|---|---|---|---|
| Time Point | | Initial | 24 h |
| Assay of Melphalan by HPLC | | 97.9 | 97.2 |
| (Unit: % of label amount) | | | |
| Related Substances | Monochloro Melphalan | 0.67 | 0.71 |
| by HPLC | Melphalan Dimer | 0.40 | 0.42 |
| (Unit: %) | Single maximum | 0.16 | 0.16 |
| | unknown impurity | | |
| | Total Impurities | 1.92 | 1.87 |

While the present invention has been described in terms of its specific embodiments, certain modifications and equivalents will be apparent to those skilled in the art and are intended to be included within the scope of the present invention.

1. A sterile injectable composition comprising melphalan and one or more phospholipids.

2. The sterile injectable composition according to claim 1, wherein the phospholipid is 1,2-dimyristoyl-sn-glycero-3-phosphorylglycerol (DMPG), N-(carbonyl-methoxypolyethylene glycol 2000)-1,2-distearoyl-sn-glycero-3-phosphoethanolamine (mPEG₂₀₀₀-DSPE), or any combination thereof.

3. The sterile injectable composition according to claim 2 further comprises one or more stabilizers.

4. The sterile injectable composition according to claim 3, wherein the stabilizer is polyvinylpyrrolidone (PVP), sucrose, or any combination thereof.

5. The sterile injectable composition according to claim 2, wherein melphalan and DMPG are present in a weight ratio between 1:1 and 1:4.

6. The sterile injectable composition according to claim 2, wherein melphalan and mPEG₂₀₀₀-DSPE are present in a weight ratio between 1:1 and 1:8.

7. The sterile injectable composition according to claim 4, wherein melphalan and the stabilizer are present in a weight ratio between 1:5 and 1:30.

8. The sterile injectable composition according to claim 1, wherein the composition is a clear aqueous solution.

9. The sterile injectable composition according to claim 8, wherein the solution further comprises one or more pH adjusting agents.

10. The sterile injectable composition according to claim 9, wherein the pH adjusting agent is present in a quantity to provide pH of the solution about 4.

11. The sterile injectable composition according to claim 8, wherein the solution has % transmittance not less than 99%, when measured at 650 nm.

12. The sterile injectable composition according to claim 8, wherein the solution has absorbance not more than 0.1 AU (absorbance unit), when measured at 420 nm.

13. The sterile injectable composition according to claim 8, wherein the solution does not form any precipitate after storage for 24 hours at 25° C.±2° C. temperature.

14. The sterile injectable composition according to claim 8, wherein the solution is lyophilizable.

15. The sterile injectable composition according to claim 8, wherein the solution retain at least 90% potency of the melphalan after storage for 4 hours at 25° C.±2° C. temperature.

16. The sterile injectable composition according to claim 8, wherein the solution retain at least 90% potency of the melphalan after storage for 4 hours at 2° C.-8° C.

17. The sterile injectable composition according to claim 1, wherein the composition is a lyophilized powder for injection.

18. The sterile injectable composition according to claim 17, wherein the lyophilized powder retain at least 90% potency of the melphalan after storage for 12 months at 25° C.±2° C. temperature and 60% RH.

19. The sterile injectable composition according to claim 17, wherein the lyophilized powder retain at least 90% potency of the melphalan after storage for 12 months at 2° C.-8° C.

20. The sterile injectable composition according to claim 17, wherein the lyophilized powder provides a clear solution when reconstituted with one or more aqueous vehicles.
